(19) United States
(12) Patent Application Publication
(10) Pub. No.: US 2008/0166339 A1
(43) Pub. Date: Jul. 10, 2008

(54) Inhibition of osteopontin for treatment of relapsing autoimmune disease

(76) Inventors: Sawsan Youssef, Palo Alto, CA (US); Eun Mi Hur, Stanford, CA (US); Lawrence Steinman, Stanford, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/999,099

(22) Filed: Dec. 3, 2007

Related U.S. Application Data
(60) Provisional application No. 60/872,076, filed on Dec. 1, 2006.

(51) Int. Cl. A61K 39/395; A61K 31/70; A61P 37/00

(52) U.S. Cl. 424/130.1; 514/43

(57) ABSTRACT

Methods are provided for the inhibition or prevention of relapses in pre-existing autoimmune disease by decreasing activity or expression of osteopontin in immune cells found in tissues affected by the autoimmune disease. Osteopontin is shown herein to mediate autoimmune relapses and induce a shift to the secondary or progressive stage in autoimmune disease. Osteopontin promotes the survival of activated T cells through regulation of transcription factors, FoxO3a and NF-κB and via the expression of pro-apoptotic proteins.

### FIELD OF THE INVENTION

This invention relates to treating and preventing relapsing autoimmune disease such as multiple sclerosis.

### BACKGROUND OF THE INVENTION

One important characteristic of autoimmune disorders, chronically afflicting up to eight percent of the population in the economically developed world, is the alternation of periods of remission and exacerbation, described as recurrent relapses. These relapses contribute to the burden of chronic disability. The mechanisms underlying the reactivation of disease, culminating in exacerbation and progression of autoimmunity remain elusive.

Osteopontin (OPN), or early T cell activation gene-1 (Eta-1), is a multifunctional protein that has been implicated in a number of physiological and pathological events, including bone remodeling, cancer and inflammation. The highly elevated expression of OPN at the site of pathology, observed in several autoimmune diseases, including multiple sclerosis (MS), systemic lupus erythematosus (SLE), rheumatoid arthritis (RA) and inflammatory bowel diseases (IBD), has focused attention on this molecule as a potentially critical factor in pathogenesis.

T cell death is pivotal in establishing self-tolerance and in preventing autoimmunity. The elimination of potentially self-reactive T cells is required both for central tolerance during thymic development and for peripheral tolerance. Despite these two layers of protection against activation of autoaggression, undesirable activation of self-reactive T cells, which escape from these regulatory mechanisms, poses a threat. Therefore, it is possible that the immune system incorporates further mechanisms to extinguish primary autoimmune attacks arising as a consequence of a break in tolerance to self. The apoptotic elimination of an already activated autoreactive T cell population after the initial autoimmune response might exist as one such mechanism for deletion of deleterious T cells that escaped elimination in the thymus or in the periphery. In fact, T cell death occurs in the CNS of mice with EAE at the time of spontaneous remission from paralytic disease.

### SUMMARY OF THE INVENTION

The invention provides methods for treating or preventing relapses in relapsing autoimmune diseases, including relapsing demyelinating diseases, such as multiple sclerosis, chronic inflammatory demyelinating polyneuropathy, etc.; relapsing rheumatoid autoimmune disease, e.g. relapsing polychondritis; and the like. The methods of the invention comprise administering to a subject having a pre-existing disease conditions an effective amount of an inhibitor of osteopontin, to suppress or prevent relapses of the disease.

In some embodiments, a method is provided for inhibiting relapses in autoimmune diseases in a subject, the method comprising administering to the subject a prophylactically effective amount of a nucleic acid that specifically reduces levels of osteopontin, e.g. an anti-sense oligonucleotide, siRNA, and the like.

In other embodiments, a method is provided for inhibiting relapses in autoimmune diseases in a subject, the method comprising administering to the subject a therapeutically effective amount of an anti-osteopontin antibody or antigen-binding portion thereof. In another embodiments, a method is provided for inhibiting relapses in autoimmune diseases in a subject, the method comprising administering to said subject an agent that downregulates the expression, or inhibits the activity of, a ligand of osteopontin, which ligands include, without limitation, CD44, RGD, and α-4 integrin.

In some methods of the invention, the subject is a human, in other methods the subject is a mouse. In some methods, the level of osteopontin is monitored in a cell of the patient selected from the group consisting of a neuron, a macrophage, a vascular endothelial cell, an astrocyte and a microglial cell. In some methods, the patient has ongoing demyelinating disease and the method further comprises monitoring a decrease in the symptoms of the patient responsive to the administering of an osteopontin inhibitor. In some embodiments of the invention, myelin-reactive or other activated T cells, particularly T cells present in CSF, are monitored for one or more of survival, expression of transcription factors, and expression of pro-apoptotic proteins, to determine, for example, if the treatment is effective in reducing the survival of such activated T cells.

It is shown herein that osteopontin (OPN), is increased in patient's plasma during relapses of multiple sclerosis. In models of MS including relapsing, progressive and multifocal experimental autoimmune encephalomyelitis (EAE), OPN triggered recurrent relapses, promoted worsening paralysis, and induced neurological deficits including optic neuritis. Increased inflammation followed OPN administration, whereas its absence resulted in more cell death of brain infiltrating lymphocytes. OPN promotes activated T cell survival by inhibiting transcription factor, FoxO3a, by activating NF-κB through induction of IKKβ phosphorylation and by altering expression of pro-apoptotic proteins, Bim, Bak and Bax. These mechanisms collectively suppress death of myelin-reactive T cells, linking OPN with the relapses and insidious progression characterizing MS.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 Opn induces worsening autoimmune relapses and severe progression of autoimmune demyelinating disease. (a) Clinical scores of EAE induced by immunization of Opn-wild-type mice (Opn-WT; n=10) and Opn-knockout mice (Opn-KO; n=14) with MOG peptide; Opn-knockout mice (n=7) were given rOPN daily for 32 d after the initial peak of the clinical disease, initiated during the first remission of each mouse. (b) Clinical scores of female SJL/J mice immunized with the peptide of proteolipid protein and then treated with PBS (n ¼ 9 mice) or with rOPN (n=9 mice) as described in a. Upward arrows (a,b), first day of rOpn treatment. (c) Clinical scores of MOG-specific TCR-transgenic mice given primary immunization with MOG peptide without pertussis toxin and then treated with PBS (n=6 mice) or with rOPN (n=6 mice) daily beginning on the day of primary immunization; mice were immunized with MOG peptide plus pertussis toxin 25 d after primary immunization. (d) Hematoxylin-and-eosin staining of optic nerve tissue sections isolated from MOG-specific TCR-transgenic mice with optic neuritis and EAE. (e) TUNEL staining of brain and spinal cord tissue from Opn-knockout and Opn-wildtype mice immunized with MOG peptide to induce EAE, obtained on day 17 after immunization. Arrows indicate TUNEL-positive (brown) nuclei of infiltrating lymphocytes stained with 3,3′-diaminobenzidine. Scale bars, 25 μm. Data represent mean clinical score (+s.e.m.) of 00 experiments (a-c) or 00 experiments (d,e).

FIG. 2 Opn inhibits cell death but does not affect cell division. (a) TUNEL assay of the death of Opn-wild-type lymph node T cells stimulated with concanavalin A and cultured for 24 h in the presence or absence of rOPN (2 μg/ml). Fluorescein isothiocyanate-stained TUNEL positive cells among stimulated T cells (solid lines) were analyzed by flow cytometry; filled histograms, unstimulated, resting T cells (control). Numbers below bracketed lines indicate TUNEL-positive cells. (b) Flow cytometry of the CFSE profiles of cultured CD4⁺ and CD8⁺ T cells. CFSE-labeled splenocytes from Opn-knockout mice were stimulated for 72 h as described in a in culture with or without rOPN (2 μg/ml); the CFSE fluorescence of unstimulated cells (solid lines) and stimulated cells (filled histograms) was analyzed after gating on CD4⁺CFSE⁺ or CD8⁺CFSE⁺ cells. Numbers below bracketed lines indicate percent dividing cells. Data are representative of three independent experiments.

FIG. 3 Opn regulates the activation of Foxo3a and NF-κB and expression of Bim, Bak and Bax. (a) Immunoblot analysis of phosphorylated (p-) PDK-1, PTEN and Akt in lysates of purified T cells activated for 48 h by immobilized anti-CD3 and anti-CD28 in the presence of rOPN. (b) Immunoblot analysis of Foxo3a phosphorylation in activated T cells stimulated by anti-CD3 plus anti-CD28 (left) or concanavalin A (ConA; right) and cultured for 48 h with rOPN. Below, quantification of immunoblot for the ratio of phosphorylated Foxo3a to total Foxo3a (p-Foxo3a/Foxo3a). (c) Immunoblot analysis of the abundance of IκBα in lysates of T cells activated as described as in a and cultured for 48 h with rOPN. Below, quantification of immunoblot for the ratio of IκBα to β-actin. (d) NF-κB (p65 and p50) DNA-binding assay of nuclear fractions of Opn-knockout and Opn-wild-type CD3⁺T cells activated for 48 h with anti-CD3 plus anti-CD28 (top) or Opn-wild-type CD3⁺T cells activated in the presence of rOPN or anti-Opn (10 μg/ml; middle and bottom). A₆₅₀, absorbance at 650 nm. (e) Immunoblot analysis of IKKβ phosphorylation in T cells activated as described in a and cultured with rOPN for 24 h. Below, quantification of immunoblot for the ratio of phosphorylated IKKβ to total IKKβ. (f) Immunoblot analysis of the expression of Bim, Bak and Bax in purified naïve Opn-wild-type (WT) and Opn-knockout (KO) CD4⁺ and CD8⁺T cells. Right, quantification of immunoblot at left for expression relative to that of β-actin: filled bars, Opn-wild-type; open bars, Opn-knockout. Results are representative of three (a,f), 00 (b), 00 (c), 00 (d) or 00 (e) independent experiments.

FIG. 4. Mode of cell death inhibited by Opn in T cell. (a) TUNEL assay of the death of Opn-wild-type or Opn-knockout lymph node T cells stimulated with concanavalin A in the presence (zVad) or absence (Mock) of z-VAD-fmk, assayed 24 h after activation; TUNEL-positive CD4. T cells were assessed by flow cytometry as described in FIG. 2a. Numbers above bracketed lines indicate TUNEL-positive cells. (b,c) Flow cytometry to assess the survival of Opn-wildtype or Opn-knockout CD4⁺ and CD8⁺ lymph node T cells either activated as described in a (b) or resting (naïve; c); cells were cultured in the presence or absence of z-VAD-fmk and were stained with anti-CD4 or anti-CD8 every 12 h, and the percent live cells was determined by propidium iodide staining at various times (horizontal axis) after activation. Data (a-c) are representative of two independent experiments.

FIG. 5. Opn promotes the survival of adoptively transferred T cells in vivo. CD3⁺ T cells were purified from the lymph nodes of Opn-knockout or Opn wild-type donor mice, then naïve or activated T cells (3×10⁶ cells) labeled with CFSE were adoptively transferred intravenously into syngeneic Rag1^{-/-} recipient mice; at day 8 after transfer, transferred CFSE⁺T cells from the spleen and lymph nodes from the recipient mice were analyzed by flow cytometry to assess survival and proliferation. (a) Percent survival (mean±s.e.m.) of CFSE⁺T cells from Opn-wild-type donors (filled bars) or Opn knockout donors (open bars) in spleen (SpI) or lymph nodes (LN) of recipient mice. n=3 mice. (b) CFSE fluorescence of naïve or activated T cells transferred from Opn-wild-type or Opn-knockout donors and isolated from lymph nodes of recipient mice. Numbers above bracketed lines indicate percent dividing cells. Data are representative of 00 experiments.

FIG. 6. Autoimmune optic neuritis induced by OPN in MOG-specific TCR transgenic mice with EAE. (a) OPN-treated and PBS-treated mice in FIG. 1c were examined daily for assessment of clinical signs including eyelid redness, swelling, tearing and atrophy of the eye. (b) Enhanced infiltration of mononuclear cells in CNS tissues with EAE by OPN administration. Hematoxylin and eosin staining of CNS tissue section isolated from MOG-specific TCR transgenic mice with optic neuritis and EAE as described in FIG. 1c. The histological images are representative of four different CNS tissues of PBS- or rOPN-treated groups each.

FIG. 7. Residual LPS in purified OPN does not affect clinical score of EAE. EAE was induced in 2D2 mice (5 per group) (a), SJL/J (5 per group) (b, c). Mice were treated with rOPN (5 μg) or LPS (0.5 ng) or PBS i.v. daily from day 0 (a and b) or from day 20 (after first remission). Arrows indicate the starting day of treatment. Data represent mean clinical score and s.e.m.

FIG. 8. Increased T cell proliferation by OPN. Splenocytes (2.5×10 /ml) from MBP TCR Transgenic mice were stimulated with a specific peptide Ac1-11 (0-10 μg/ml) and cultured with 0 (open circle), 0.4 (open diamond) and 4 (closed circle) μg/ml of soluble recombinant OPN (rOPN). [H]-thymidine was added to the triplicates (n=3, mean±s.e.m.), after 96 h of antigen stimulation; its incorporation was measured after 18 h.

FIG. 9. The time course expression of Bim, Bak and Bax after T cell activation in vitro. (a) Cells isolated from LN and spleen of the OPN-WT or OPN-KO mice were stimulated with ConA (2 μg/ml) and cultured for the indicated time. After T cell activation, whole cell lysates (1×10 cells per lane) from the culture were subjected to immunoblot analysis. Data are representative of three independent experiments. (b) Expression profile of pro-apoptotic Bcl-2 family proteins after T cell activation in OPN-WT and OPN-KO cells. Graphs represent relative expression, based on the intensity of signals that were normalized by β actin abundance in immunoblot analysis. Relative expression=(Iₜ−min)/(maxmin)×100. The Iₜ: signal intensity of the corresponding protein at the time point; the min: signal intensity of the protein when it exhibits the lowest signal during the time course; the max: signal intensity of the protein when it exhibits the highest signal during the time period.

FIG. 10. OPN does not affect Fas-induced cell death. Cells isolated from the LN and spleen of the OPN-WT (closed circle) or OPN-KO (open circle) mice (n=3) were stimulated with ConA (2 μg/ml) and cultured for three days. Stimulated cells were crosslinked with anti-Fas antibody (1 μg/ml) and cultured for the indicated times. Cell survival was quantified by staining with PI and anti-CD4 or anti-CD8 and flow cytometric analysis. Data are representative of two independent experiments.

FIG. 11. OPN inhibits nuclear translocation of AIF. (a) Dysregulated nuclear translocation of Apoptosis inducing factor (AIF) in OPN-KO splenocytes. Splenocytes isolated from OPN-WT and OPN-KO mice were stimulated with ConA (2 μg/ml) and cultured for the indicated times. At each time point, cells from the culture were lysed and subject to subcellular fractionation. Nuclear (Nuc) and mitochondrial (Mit) fractions were obtained by differential centrifugation. Mouse lymph node cells and splenocytes (5×10 cells) were used. The nuclear translocation of AIF was assessed by an immunoblot of proteins from mitochondrial and nuclear fractions; Histone (H3) was used as a nuclear marker protein. Data are representative of four independent experiments. (b) Translocation profile of AIF after T cell activation in OPN-WT and OPN-KO cells. Graphs represent the relative expression of AIF in nuclear and mitochondrial fractions, as described in Supplementary FIG. 2c. (c) rOPN inhibits nuclear translocation of AIF upon T cell activation. LN T cells isolated from OPN-WT mice were activated and cultured for 48 h with different concentrations of rOPN (0-10 μg/ml). Cells from the culture were lysed and subject to subcellular fractionation as in (b). The AIF abundance in the nucleus was determined using immunoblot analysis. The bar graph represents the ratio of nuclear AIF to H3 assessed from the intensity of the signals obtained from the immunoblot analysis.

FIG. 12. Schematic diagram of OPN-induced survival of T cell. OPN induces phosphorylation and retention in cytosol of FoxO3a. NF-κB activation is also induced by OPN. The inhibition of FoxO3a along with activation of NF-κB may result in suppression of anti-survival proteins whereas induction of pro-survival proteins. The expression of anti-survival Bcl-2 family proteins, Bim, Bak and Bax is altered by OPN. Translocation of AIF to nucleus from mitochondria, where AIF plays role as a pro-survival protein, is inhibited by OPN.

FIG. 13. Schematic diagram of the suggested model for autoimmune relapse. Autoreactive T cells can be activated as the result of a tolerance break and cause autoimmune responses through clonal expansion in a way similar to that of a non-self antigen. Subsequently, activated autoimmune T cells might undergo programmed cell death, resulting in the clearance of pathogenic autoreactive T cells, which is a process suggested as ‘Post-immune extermination (PIE)’. PIE might be responsible for the remission of an autoimmune attack. OPN might promote activated T cells escaping from PIE and contraction by inhibiting programmed cell death. The failure or leakage of PIE might lead to the enhanced survival of autoreactive T cells and cause recurrent autoimmune relapses.

### DETAILED DESCRIPTION

### Definitions

“Activity” of osteopontin shall mean any enzymatic or binding function performed by that protein. Osteopontin activity includes, for example, binding to CD44.

“Antibody” shall include, by way of example, both naturally occurring and non-naturally occurring antibodies. Specifically, this term includes polyclonal and monoclonal antibodies, and fragments thereof. Furthermore, this term includes chimeric antibodies and wholly synthetic antibodies, and fragments thereof.

“Anti-sense nucleic acid” shall mean any nucleic acid which, when introduced into a cell, specifically hybridizes to at least a portion of an mRNA in the cell encoding a protein (“target protein”) whose expression is to be inhibited, and thereby inhibits the target protein's expression.

“Catalytic nucleic acid” shall mean a nucleic acid that specifically recognizes a distinct substrate and catalyzes the chemical modification of this substrate.

“Comparable cell” shall mean a cell whose type is identical to that of another cell to which it is compared. Examples of comparable cells are cells from the same cell line.

“DNAzyme” shall mean a catalytic nucleic acid that is DNA or whose catalytic component is DNA, and which specifically recognizes and cleaves a distinct target nucleic acid sequence, which can be either DNA or RNA. Each DNAzyme has a catalytic component (also referred to as a “catalytic domain”) and a target sequence-binding component consisting of two binding domains, one on either side of the catalytic domain. [0075] “Endogenous protein” shall mean, with respect to a particular subject, a protein originally encoded by the subject's own genome.

“Expressible nucleic acid” shall mean a nucleic acid encoding a nucleic acid of interest and/or a protein of interest which nucleic acid is an expression vector, plasmid or other construct which, when placed in a cell, permits the expression of the nucleic acid or protein of interest. Expression vectors and plasmids are well known in the art.

“Inhibiting” the onset of a disorder shall mean either lessening the likelihood of the disorder's onset, or preventing the onset of the disorder entirely. In the preferred embodiment, inhibiting the onset of a disorder means preventing its onset entirely. As used herein, onset may refer to a relapse in a patient that has ongoing relapsing remitting disease.

The methods of the invention are specifically applied to patients that have been diagnosed with an autoimmune disease, e.g. a relapsing-remitting disease. Treatment is aimed at the treatment or prevention of relapses, which are an exacerbation of a pre-existing condition.

“Inhibiting” the expression of a gene in a cell shall mean either lessening the degree to which the gene is expressed, or preventing such expression entirely.

“Nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

“Osteopontin” shall mean the human protein encoded by the mRNA sequence set forth in GenBank Accession No. J04765, all naturally occurring variants and homologues thereof, and where applicable herein, all antigenic fragments thereof.

Active fragments of osteopontin share a functional or binding property with full length osteopontin.

Epitopic fragments of osteopontin bind to a monoclonal antibody that binds to full length osteopontin.

“Osteopontin-related disorder” shall mean any disorder (a) characterized by the over-expression of osteopontin in an afflicted subject, (b) ameliorated by inhibiting osteopontin expression in an afflicted subject, and/or (c) ameliorated by inhibiting osteopontin activity in an afflicted subject, (d) in which expression of osteopontin contributes to the pathogenesis.

Expression of osteopontin that is normal in some individuals may nevertheless contribute toward an osteopontin-related disorder in other individuals if such other individuals the osteopontin acts in combinations with another cellular component, such as a protein, in pathogenesis. Some osteopontin-related disorders are characterized by an elevated Th1 immune response and a depressed Th2 immune response relative to the mean of such responses in a population of normal individuals (i.e., free of an osteopontin-related disease and not at risk of such a disease).

Over-expression of osteopontin means an expression level that is greater than the mean plus one standard deviation of that in a population of normal individuals. Preferably the expression level is at least ten times the mean of that in a population of normal individuals.

“Ribozyme” shall mean a catalytic nucleic acid molecule which is RNA or whose catalytic component is RNA, and which specifically recognizes and cleaves a distinct target nucleic acid sequence, which can be either DNA or RNA. Each ribozyme has a catalytic component (also referred to as a “catalytic domain”) and a target sequence-binding component consisting of two binding domains, one on either side of the catalytic domain.

“Specifically hybridize” to a nucleic acid shall mean, with respect to a first nucleic acid, that the first nucleic acid hybridizes to a second nucleic acid with greater affinity than to any other nucleic acid.

“Specifically inhibit” the expression of a protein shall mean to inhibit that protein's expression (a) more than the expression of any other protein, or (b) more than the expression of all but 10 or fewer other proteins.

“Subject” or “patient” shall mean any animal, such as a human, non-human primate, mouse, rat, guinea pig or rabbit.

“Suitable conditions” shall have a meaning dependent on the context in which this term is used. That is, when used in connection with an antibody, the term shall mean conditions that permit an antibody to bind to its corresponding antigen. When this term is used in connection with nucleic acid hybridization, the term shall mean conditions that permit a nucleic acid of at least 15 nucleotides in length to hybridize to a nucleic acid having a sequence complementary thereto. When used in connection with contacting an agent to a cell, this term shall mean conditions that permit an agent capable of doing so to enter a cell and perform its intended function. In one embodiment, the term “suitable conditions” as used herein means physiological conditions.

“Treating” a disorder shall mean slowing, stopping or reversing the disorder's progression. In the preferred embodiment, treating a disorder means reversing the disorder's progression, ideally to the point of eliminating the disorder itself. As used herein, ameliorating a disorder and treating a disorder are equivalent.

The term “immune” response is the development of a beneficial humoral (antibody mediated) and/or a cellular (mediated by antigen-specific T cells or their secretion products) response directed against osteopontin an amyloid peptide in a recipient patient. Such a response can be an active response induced by An “immunogen” is capable of inducing an immunological response against itself on administration to a mammal, optionally in conjunction with an adjuvant.

The term “naked polynucleotide” refers to a polynucleotide not complexed with colloidal materials. Naked polynucleotides are sometimes cloned in a plasmid vector.

The term “adjuvant” refers to a compound that when administered in conjunction with an antigen augments the immune response to the antigen, but when administered alone does not generate an immune response to the antigen. Adjuvants can augment an immune response by several mechanisms including lymphocyte recruitment, stimulation of B and/or T cells, and stimulation of macrophages.

Unless otherwise apparent from the context, all elements, steps or features of the invention can be used in any combination with other elements, steps or features.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

The subject methods are used for prophylactic or therapeutic purposes. As used herein, the term “treating” is used to refer to both prevention of relapses, and treatment of on ongoing relapse. For example, the prevention of a relapse may be accomplished by administration of the agent prior to development of a relapse. The treatment of ongoing disease, where the treatment stabilizes or improves the clinical symptoms of the patient, is of particular interest.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

### METHODS OF THE INVENTION

The present invention provides methods for preventing relapses in autoimmune disease, including relapsing demyelinating diseases, such multiple sclerosis; relapsing rheumatoid disease, etc. These methods comprise administering to the subject diagnosed as having an existing autoimmune condition, e.g. a relapsing remitting condition; an effective amount of an inhibitor of osteopontin.

In some embodiments, a method is provided for inhibiting relapses in autoimmune diseases in a subject, the method comprising administering to the subject a prophylactically effective amount of a nucleic acid that specifically reduces levels of osteopontin, e.g. an anti-sense oligonucleotide, siRNA, and the like.

In other embodiments, a method is provided for inhibiting relapses in relapsing autoimmune diseases in a subject, the method comprising administering to the subject a therapeutically effective amount of an anti-osteopontin antibody or antigen-binding portion thereof.

In another embodiments, the method comprising administering to said subject an agent that downregulates the expression, or inhibits the activity of, a ligand of osteopontin, which ligands include, without limitation, CD44, RGD, and α-4 integrin. In these methods, the osteopontin-expressing cell can be, without limitation, a neuron, a macrophage, a vascular endothelial cell, an astrocyte or a microglial cell.

This invention can utilize a method for reducing the amount of osteopontin in an osteopontin-expressing cell comprising introducing into the cell a nucleic acid which specifically inhibits osteopontin expression in the cell. In one embodiment, this method further reduces the amount of osteopontin secreted by an osteopontin-secreting cell. In this method, the nucleic acid can be, for example, DNA or RNA. In addition, the nucleic acid can be an anti-sense nucleic acid that hybridizes to osteopontin-encoding mRNA, an siRNA that inhibits osteopontin expression, or a catalytic nucleic acid that cleaves osteopontin-encoding mRNA. Osteopontin expression can also be inhibited using zinc finger proteins or nucleic acids encoding the same as described in WO 00100409. Alternatively, inhibition of expression can be achieved using siRNAs as described by WO 99132619, Elbashir, EMBO J. 20, 6877-6888 (2001) and Nykanen et al., Cell 107, 309-321 (2001); WO01129058.

Alternatively, autoimmune relapse in a subject is treated by administering to the subject a therapeutically effective amount of a nucleic acid that specifically inhibits the expression of osteopontin in the subject's osteopontin-expressing cells.

In these methods of prophylaxis and treatment, the nucleic acid can be, for example, DNA or RNA. In addition the nucleic acid can be an anti-sense nucleic acid that hybridizes to osteopontin-encoding mRNA, an siRNA, a catalytic nucleic acid that cleaves osteopontin-encoding mRNA, etc.

In another embodiment, relapse of an autoimmune disease in a subject is inhibited or prevented by administering to the subject a prophylactically or therapeutically effective amount of an anti-osteopontin antibody or antigen-binding portion thereof.

Determining a therapeutically or prophylactically effective amount of the osteopontin inhibitor compositions can be done based on animal data using routine computational methods. In one embodiment, the therapeutically or prophylactically effective amount contains between about 0.1 mg and about 1 g of nucleic acid or protein, as applicable. In another embodiment, the effective amount contains between about 1 mg and about 100 mg of nucleic acid or protein, as applicable. In a further embodiment, the effective amount contains between about 10 mg and about 50 mg of the nucleic acid or protein, as applicable.

In this invention, administering the instant compositions can be effected or performed using any of the various methods and delivery systems known to those skilled in the art. The administering can be performed, for example, intravenously, orally, via implant, transmucosally, transdermally, intramuscularly, intrathecally, and subcutaneously. The following delivery systems, which employ a number of routinely used pharmaceutical carriers, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone. Osteopontin or nucleic acids of the invention can also be administered attached to particles using a gene gun.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, xanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and Jun. 2, 2005 antioxidants (e.g., parabens, vitamins E and C, and ascorbic acid), anti-caking agents, coating agents, and chelating agents (e.g., EDTA).

### Conditions for Analysis and Therapy

The compositions and methods of the invention find use in combination with a variety of relapsing autoimmune conditions, including relapsing demyelinating autoimmune diseases. Generally patients for the methods of the present invention are diagnosed as having an autoimmune condition, e.g. a relapsing-remitting autoimmune condition, prior to treatment. The inhibition of osteopontin decreases the severity or incidence of relapses in such patients.

Multiple sclerosis (MS) is characterized by various symptoms and signs of CNS dysfunction, with remissions and recurring exacerbations. The most common presenting symptoms are paresthesias in one or more extremities, in the trunk, or on one side of the face; weakness or clumsiness of a leg or hand; or visual disturbances, e.g. partial blindness and pain in one eye (retrobulbar optic neuritis), dimness of vision, or scotomas. Other common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances;. all indicate scattered CNS involvement and often occur months or years before the disease is recognized. Excess heat may accentuate symptoms and signs.

Clinical data alone may be sufficient for a diagnosis of MS. If an individual has suffered two separate episodes of neurologic symptoms characteristic of MS, and the individual also has consistent abnormalities on physical examination, a diagnosis of MS can be made with no further testing. Magnetic resonance imaging (MRI) of the brain and spine is often used during the diagnostic process. MRI shows areas of demyelination (lesions) as bright spots on the image. A substance, called Gadolinium, can be injected into the spinal column to highlight active plaques and, by elimination, demonstrate the existence of historical lesions not associated with clinical symptoms. This can provide the evidence of chronic disease needed for a definitive diagnosis of MS. Testing of cerebrospinal fluid (CSF) can provide evidence of chronic inflammation of the central nervous system. The CSF is tested for oligoclonal bands, which are immunoglobulins found in 85% to 95% of people with definite MS. Combined with MRI and clinical data, the presence of oligoclonal bands can help make a definite diagnosis of MS. Lumbar puncture is the procedure used to collect a sample of CSF.

The brain of a person with MS often responds less actively to stimulation of the optic nerve and sensory nerves. These brain responses can be examined using visual evoked potentials (VEPs) and somatosensory evoked potentials (SEPs). Decreased activity on either test can reveal demyelination which may be otherwise asymptomatic. Along with other data, these exams can help find the widespread nerve involvement required for a definite diagnosis of MS.

In 1996 the United States National Multiple Sclerosis Society standardized the following four subtype definitions (see Lublin and Reingold (1996) Neurology 46(4):907-11, herein specifically incorporated by reference) as relapsing-remitting; secondary progressive; primary progressive; progressive relapsing. The methods of the invention find particular use in the treatment of ongoing disease, and particularly in treating relapsing forms.

Relapsing-remitting describes the initial course of 85% to 90% of individuals with MS. This subtype is characterized by unpredictable attacks (relapses) followed by periods of months to years of relative quiet (remission) with no new signs of disease activity. Deficits suffered during the attacks may either resolve or may be permanent. When deficits always resolve between attacks, this is referred to as “benign” MS.

Secondary progressive describes around 80% of those with initial relapsing-remitting MS, who then begin to have neurologic decline between their acute attacks without any definite periods of remission. This decline may include new neurologic symptoms, worsening cognitive function, or other deficits. Secondary progressive is the most common type of MS and causes the greatest amount of disability.

Primary progressive describes the approximately 10% of individuals who never have remission after their initial MS symptoms. Decline occurs continuously without clear attacks. The primary progressive subtype tends to affect people who are older at disease onset.

Progressive relapsing describes those individuals who, from the onset of their MS, have a steady neurologic decline but also suffer superimposed attacks; and is the least common of all subtypes.

Treatments for MS include interferon β (Avonex, Betaseron, Rebif), Copaxone (Glatiramer acetate), and anti-VLA4 (Tysabri, natalizumab), which reduce relapse rate and to date have only exhibited a modest impact on disease progression. MS is also treated with immunosuppressive agents including methylprednisolone, other steroids, methotrexate, cladribine and cyclophosphamide. Many biological agents, such as anti-IFNgamma antibody, CTLA4-Ig (Abetacept), anti-CD20 (Rituxan), and other anti-cytokine agents are in clinical development for MS.

Peripheral neuropathies may also have a relapsing remitting course, and may include Miller Fisher syndrome; chronic inflammatory demyelinating polyneuropathy (CIDP) with its subtypes classical CIDP, CIDP with diabetes, CIDP/monoclonal gammopathy of undetermined significance (MGUS), sensory CIDP, multifocal motor neuropathy (MMN), multifocal acquired demyelinating sensory and motor neuropathy or Lewis-Sumner syndrome, multifocal acquired sensory and motor neuropathy, and distal acquired demyelinating sensory neuropathy; IgM monoclonal gammopathies with its subtypes Waldenstrom's macroglobulinemia, myelin-associated glycoprotein-associated gammopathy, polyneuropathy, organomegaly, endocrinopathy, M-protein, skin changes syndrome, mixed cryoglobulinemia, gait ataxia, late-onset polyneuropathy syndrome, and MGUS.

Rheumatoid Arthritis is a chronic syndrome characterized by usually symmetric inflammation of the peripheral joints, potentially resulting in progressive destruction of articular and periarticular structures, with or without generalized manifestations. The cause is unknown. A genetic predisposition has been identified and, in white populations, localized to a pentapeptide in the HLA-DR beta1 locus of class II histocompatibility genes. Environmental factors may also play a role. Immunologic changes may be initiated by multiple factors. About 0.6% of all populations are affected, women two to three times more often than men. Onset may be at any age, most often between 25 and 50 yr.

Relapsing polychondritis (RPC) is a rare rheumatic autoimmune disorder that causes inflammatory lesions in cartilage and connective tissue, primarily cartilage in the ear, eyes, and trachea. Tissue inflammation in these collagen-rich tissues is known as chondritis, whereas the term polychondritis signifies that multiple locations in the body are eventually affected. Relapsing refers to the episodic nature of this disorder. Periods of remission are interrupted by acute episodes of active disease. Symptoms also vary in severity, usually becoming progressive over time.

RPC is classified as a systemic disorder because various tissues located in different parts of the body may be affected. In most cases, RPC affects and has the potential to destroy cartilaginous tissues in the ears, nose, larynx, joints, pulmonary (lungs) bronchi, ribs and the trachea. Other organs and tissues may also be affected, including the eyes, heart, kidney, blood vessels, and central nervous system. RPC usually occurs alone although it may occur in patients with generalized vasculitis (inflammation of blood vessels) or in patients with other autoimmune disorders.

Arthritis in RPC tends to affect the small joints in the ribs, hands, feet, chest and spine, and it often resembles rheumatoid arthritis or ankylosing spondylitis although it is not erosive. Patients with cardiac involvement may show signs of mitral valve regurgitation. Most patients develop dermatological changes, most prominently mouth sores or skin ulcerations. Less frequently patients may develop nervous system symptoms, such as headache, seizures, or cognitive changes.

### Methods of Treatment Using Osteopontin Specific Inhibitors

### Inhibitors of Osteopontin

An inhibitory agent may inhibit the activity of osteopontin by a variety of different mechanisms. In certain embodiments, the inhibitory agent is one that binds to the protein osteopontin and, in doing so, inhibits its activity. In other embodiments, the inhibitory agent prevents expression or secretion of osteopontin.

Representative osteopontin inhibitory agents include, but are not limited to: antisense oligonucleotides; antibodies; and the like. Other agents of interest include, but are not limited to: naturally occurring or synthetic small molecule compounds of interest, which include numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing appropriate screening protocols.

The inhibitory agent may act on osteopontin mRNA to inhibit the activity of the target osteopontin by reducing the amount of osteopontin RNA present in the targeted cells, where the target cell may be present in vitro or in vivo. By “reducing the amount of” is meant that the level or quantity of the target osteopontin in the target cell is reduced by at least about 2-fold, usually by at least about 5-fold, e.g., 10-fold, 15-fold, 20-fold, 50-fold, 100-fold or more, as compared to a control, i.e., an identical target cell not treated according to the subject methods.

An antisense reagent may be antisense oligonucleotides (ODN), particularly synthetic ODN having chemical modifications from native nucleic acids, or nucleic acid constructs that express such antisense molecules as RNA. The antisense sequence is complementary to the targeted mRNA, and inhibits its expression. One or a combination of antisense molecules may be administered, where a combination may comprise multiple different sequences.

Antisense molecules may be produced by expression of all or a part of the target osteopontin sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 25, usually not more than about 23-22 nucleotides in length, where the length is governed by efficiency of inhibition, specificity, including absence of cross-reactivity, and the like.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature that alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The alpha.-anomer of deoxyribose may be used, where the base is inverted with respect to the natural .beta.-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Anti-sense molecules of interest include antagomir RNAs, e.g. as described by Krutzfeldt et al., supra., herein specifically incorporated by reference. Small interfering double-stranded RNAs (siRNAs) engineered with certain ‘drug-like’ properties such as chemical modifications for stability and cholesterol conjugation for delivery have been shown to achieve therapeutic silencing of an endogenous gene in vivo. To develop a pharmacological approach for silencing mRNAs in vivo, chemically modified, cholesterol-conjugated single-stranded RNA analogues complementary to mRNAs were developed, termed ‘antagomirs’. Antagomir RNAs may be synthesized using standard solid phase oligonucleotide synthesis protocols. The RNAs are conjugated to cholesterol, and may further have a phosphorothioate backbone at one or more positions.

Also of interest in certain embodiments are RNAi agents. In representative embodiments, the RNAi agent targets the precursor molecule of the microRNA, known as pre-microRNA molecule. By RNAi agent is meant an agent that modulates expression of microRNA by a RNA interference mechanism. The RNAi agents employed in one embodiment of the subject invention are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. Where the RNA agent is a duplex structure of two distinct ribonucleic acids hybridized to each other, e.g., an siRNA, the length of the duplex structure typically ranges from about 15 to 30 bp, usually from about 15 to 29 bp, where lengths between about 20 and 29 bps, e.g., 21 bp, 22 bp, are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides. The weight of the RNAi agents of this embodiment typically ranges from about 5,000 daltons to about 35,000 daltons, and in many embodiments is at least about 10,000 daltons and less than about 27,500 daltons, often less than about 25,000 daltons.

dsRNA can be prepared according to any of a number of methods that are known in the art, including in vitro and in vivo methods, as well as by synthetic chemistry approaches. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enable one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety).

In certain embodiments, instead of the RNAi agent being an interfering ribonucleic acid, e.g., an siRNA or shRNA as described above, the RNAi agent may encode an interfering ribonucleic acid, e.g., an shRNA, as described above. In other words, the RNAi agent may be a transcriptional template of the interfering ribonucleic acid. In these embodiments, the transcriptional template is typically a DNA that encodes the interfering ribonucleic acid. The DNA may be present in a vector, where a variety of different vectors are known in the art, e.g., a plasmid vector, a viral vector, etc.

In another embodiment, the osteopontin inhibitor is an antibody. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The term includes monoclonal antibodies, multispecific antibodies (antibodies that include more than one domain specificity), human antibody, humanized antibody, and antibody fragments with the desired biological activity.

Polyclonal antibodies can be raised by a standard protocol by injecting a production animal with an antigenic composition, formulated as described above. See, e.g., Harlow and Lane, Antibodies: A Laboratory Manual, Cold Spring Harbor Laboratory, 1988. In one such technique, a Class II targetantigen comprising an antigenic portion of the polypeptide is initially injected into any of a wide variety of mammals (e.g., mice, rats, rabbits, sheep or goats). When utilizing an entire protein, or a larger section of the protein, antibodies may be raised by immunizing the production animal with the protein and a suitable adjuvant (e.g., Fruend's, Fruend's complete, oil-in-water emulsions, etc.) Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line.

In addition, the antibodies or antigen binding fragments may be produced by genetic engineering. In this technique, as with the standard hybridoma procedure, antibody-producing cells are sensitized to the desired antigen or immunogen. The messenger RNA isolated from the immune spleen cells or hybridomas is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones, which co-express a heavy and light chain (resembling the Fab fragment or antigen binding fragment of an antibody molecule). The vectors that carry these genes are co-transfected into a host (e.g. bacteria, insect cells, mammalian cells, or other suitable protein production host cell). When antibody gene synthesis is induced in the transfected host, the heavy and light chain proteins self-assemble to produce active antibodies that can be detected by screening with the antigen or immunogen.

Antibodies with a reduced propensity to induce a violent or detrimental immune response in humans (such as anaphylactic shock), and which also exhibit a reduced propensity for priming an immune response which would prevent repeated dosage with the antibody therapeutic are preferred for use in the invention. Thus, humanized, single chain, chimeric, or human antibodies, which produce less of an immune response when administered to humans, are preferred for use in the present invention. Also included in the invention are multi-domain antibodies.

A chimeric antibody is a molecule in which different portions are derived from different animal species, for example those having a variable region derived from a murine mAb and a human immunoglobulin constant region. Techniques for the development of chimeric antibodies are described in the literature. See, for example, Morrison et al. (1984) Proc. Natl. Acad. Sci. 81:6851-6855; Neuberger et al. (1984) Nature 312:604-608; Takeda et al. (1985) Nature 314:452-454. Single chain antibodies are formed by linking the heavy and light chain fragments of the Fv region via an amino acid bridge, resulting in a single chain polypeptide. See, for example, Huston et al., Science 242:423-426; Proc. Natl. Acad. Sci. 85:5879-5883; and Ward et al. Nature 341:544-546.

Antibody fragments that recognize specific epitopes may be generated by techniques well known in the field. These fragments include, without limitation, F(ab′)₂ fragments, which can be produced by pepsin digestion of the antibody molecule, and Fab fragments, which can be generated by reducing the disulfide bridges of the F(ab′)₂ fragments.

Alternatively, single chain antibodies (Fv, as described below) can be produced from phage libraries containing human variable regions. See U.S. Pat. No. 6,174,708. Intrathecal administration of single-chain immunotoxin, LMB-7 [B3(Fv)-PE38], has been shown to cure of carcinomatous meningitis in a rat model. Proc Natl. Acad. Sci USA 92, 2765-9, all of which are incorporated by reference fully herein.

In addition to entire immunoglobulins (or their recombinant counterparts), immunoglobulin fragments comprising the epitope binding site (e.g., Fab′, F(ab′)₂, or other fragments) are useful as antibody moieties in the present invention. Such antibody fragments may be generated from whole immunoglobulins by ficin, pepsin, papain, or other protease cleavage. “Fragment,” or minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif).

Candidate antibodies can be tested for by any suitable standard means, e.g. ELISA assays, etc. As a first screen, the antibodies may be tested for binding against the immunogen. After selective binding is established, the candidate antibody may be tested for appropriate activity in an in vivo model. In a preferred embodiment, antibody compounds may be screened using a variety of methods in vitro and in vivo. These methods include, but are not limited to, methods that measure binding affinity to a target, biodistribution of the compound within an animal or cell, or compound mediated cytotoxicity. These and other screening methods known in the art provide information on the ability of a compound to bind to, modulate, or otherwise interact with the specified target and are a measure of the compound's efficacy.

Anti-osteopontin antibodies may be administered daily, semi-weekly, weekly, semi-monthly, monthly, etc., at a dose of from about 0.01 mg, from about 0.1 mg, from about 1 mg, from about 5 mg, from about 10 mg, from about 100 mg or more per kilogram of body weight when administered systemically. Smaller doses may be utilized in localized administration, e.g. in direct administration to ocular nerves, etc. Humanized, chimeric human, or human antibodies are preferred for administering to human patients.

Other methods relating to the involvement of osteopontin and autoimmune disease may be found in co-pending patent application Ser. No. 10/495,893, published as US 2005/0119204, herein specifically incorporated by reference.

This invention will be better understood by reference to the Examples which follow, but those skilled in the art will readily appreciate that the information detailed is only illustrative of the invention as described more fully in the claims which follow thereafter.

### Experimental

OPN induces relapse and enhances progression of autoimmunity. Since elevated OPN expression has been reported in many autoimmune diseases including MS, SLE and RA, we attempted to elucidate the role of OPN in an autoimmune disease model. The various EAE models of MS are of particular interest in this regard because our previous study revealed that OPN-KO mice with EAE have spontaneous remissions after the initial attack of paralysis in EAE.

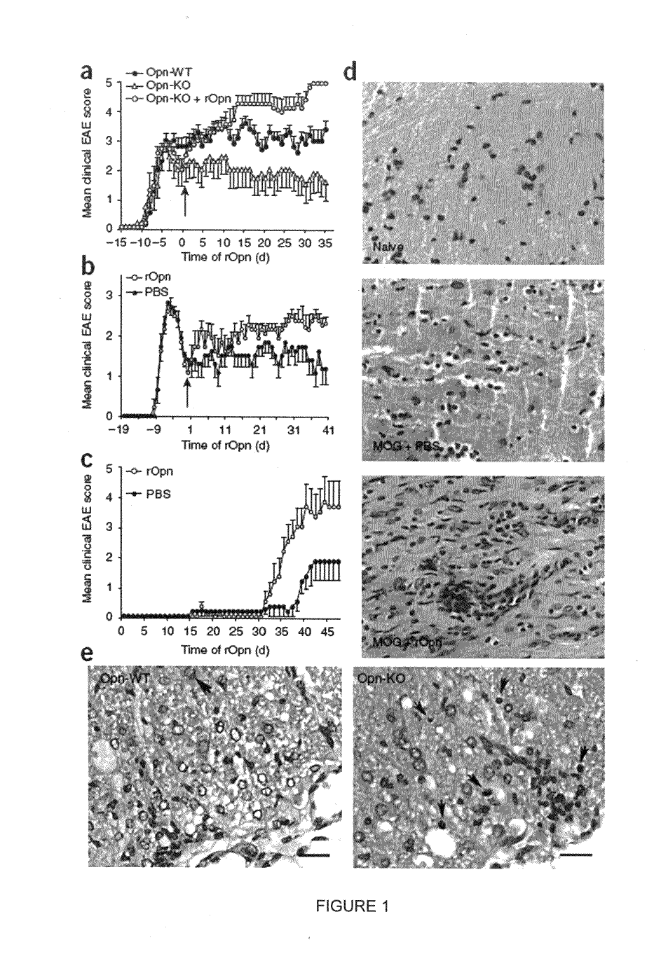

We induced acute EAE in OPN-KO and OPN-WT mice, using myelin oligodendrocyte glycoprotein (MOG) peptide 35-55 amino acids. To test whether OPN can actively exacerbate clinical paralysis, recombinant OPN (rOPN) was administered to OPN-KO mice with EAE. Injections started during the first spontaneous recovery of the disease in each individual OPN-KO mouse, defined by a decrease in clinical score for two to four consecutive days after the first peak of the disease. To mimic the condition observed in MS patients who have highly elevated plasma concentrations of OPN prior to a clinical relapse of their disease, rOPN was given intravenously to the mice daily. Strikingly, the ongoing remission of the disease was reversed and the extent of clinical paralysis worsened over time following the rOPN administration. The recrudescence of EAE induced following administration of OPN was followed by a progression to severe disease, ultimately leading to death. All OPN-treated mice (7 out of 7) died from EAE within 35 days of the administration (FIG. 1a). These results indicate that OPN inhibits spontaneous recovery and directly mediates clinical exacerbation and progression.

We next examined the effect of administration of OPN in a relapsing-remitting model of EAE, using SJL/J mice immunized with proteolipid protein (PLP) peptide (amino acids 139-151). Intravenously administered rOPN, injected during the first recovery as described above, induces rapid relapse with an increased degree of paralysis and corresponding increase in clinical score from that observed during remission. Overall there was a worsening of the clinical course with a gradual increase of average minimal and maximal scores during successive cycles of relapse and remission in this model (FIG. 1b). The fraction of animals with complete recovery, in which the clinical score returns to zero, was decreased by OPN treatment in every time period. The inhibition of spontaneous recovery by OPN in relapsing EAE was evident as early as one day following administration of OPN and was observable for 15 days. In this period, in the OPN treated group, fewer mice showed complete recovery (33%) than in the PBS-treated group (55.6%). During a later time period, from day 31 to day 45 of treatment, 8 out of 9 (88.9%) of mice in the PBS-treated group had still recovered completely whereas in the OPN treated group only 1 of 9 mice had recovered. This finding suggests that OPN induces a gradual shift from a relapsing-remitting stage to the chronic progressive stage (Table 1).

TABLE 1
EAE induced in SJL/J mice with rOPN administration

| | % Complete recovery^{b} | | | Mean minimal score^{c} | | | | Mean maximal score^{c} | | |
|---|---|---|---|---|---|---|---|---|---|---|
| | Days after treatment | | | Days after treatment | | | | Days after treatment | | |
| Treatment | 1-15 | 16-30 | 31-45 | 0 | 1-15 | 16-30 | 31-45 | 1-15 | 16-30 | 31-45 |
| rOPN^{a} | 33.3% | †11.1% | ‡11.1% | 1.1 ± 0.3 | 1.4 ± 0.4 | 1.7 ± 0.3* | 1.9 ± 0.3*** | 2.2 ± 0.2 | 2.7 ± 0.2** | 3.0 ± 0.2** |
| | (3/9)^{d} | (1/9) | (1/9) | | | | | | | |
| PBS | 55.6% | †66.7% | ‡88.9% | 1.4 ± 0.3 | 0.9 ± 0.4 | 0.7 ± 0.3* | 0.2 ± 0.2*** | 2.0 ± 0.0 | 2.1 ± 0.1** | 2.4 ± 0.2** |
| | (5/9) | (6/9) | (8/9) | | | | | | | |

^{a}recombinant (r)OPN 5 μg/mouse daily
^{b}Complete recovery is defined as the status when the clinical score is decreased to zero.
Fisher exact probability test, †p = 0.025 ‡p = 0.0017
^{c}Data are mean ± s.e.m. ANOVA test, *p < 0.1 **p < 0.05 ***p < 0.0005
^{d}(number of mice showed complete recovery in indicated time period/n)

We also tested whether the initiation of EAE by repeated activation of autoreactive T cells is augmented in the animals that receive rOPN. To do this, transgenic mice expressing MOG-specific T cell receptors (TCR), were given a primary and secondary immunization with the cognate MOG peptide 35-55, and in concert rOPN was intravenously administered daily from the initial day of primary immunization. The MOG-specific TCR transgenic mice (2D2) treated with rOPN developed more severe and fulminant EAE compared to the PBS-treated control group. This more severe clinical score was associated with a higher mortality (66.7% vs. 0%) and accelerated onset (day 9 vs. day 14) (FIG. 1c), and more severe CNS inflammation (FIG. 1d).

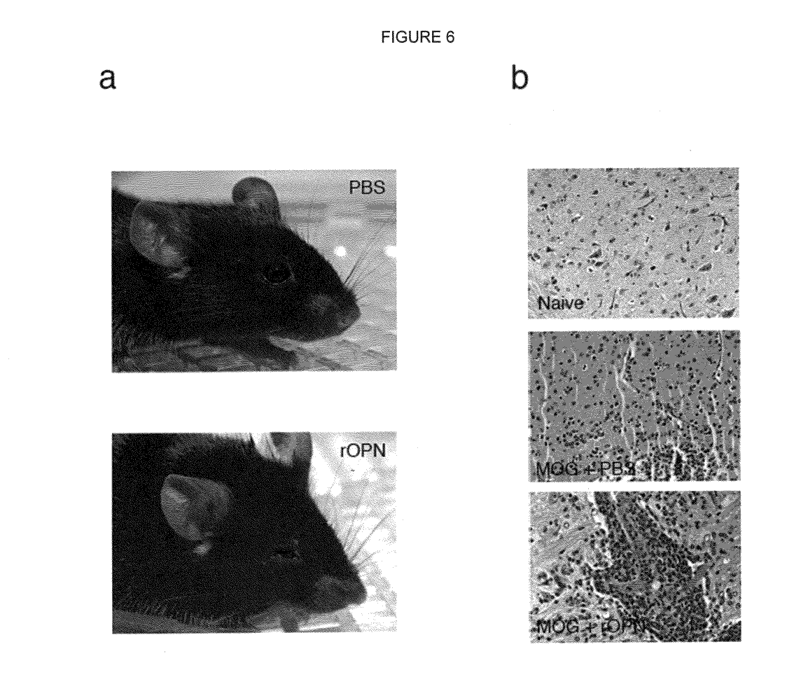

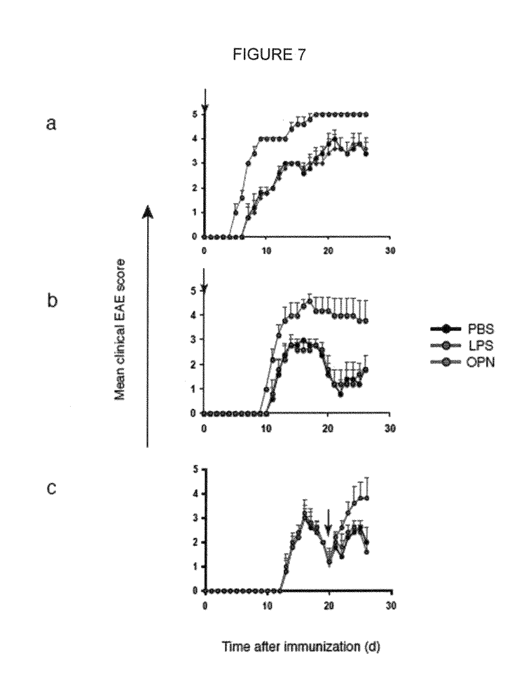

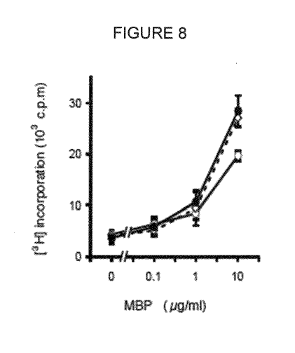

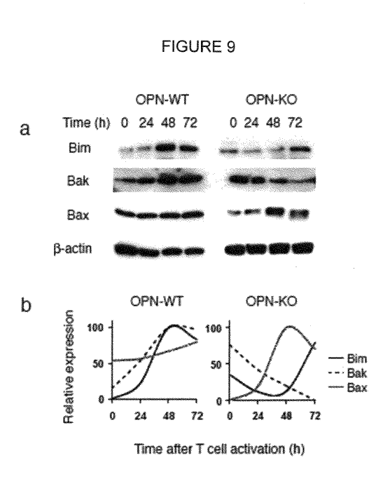

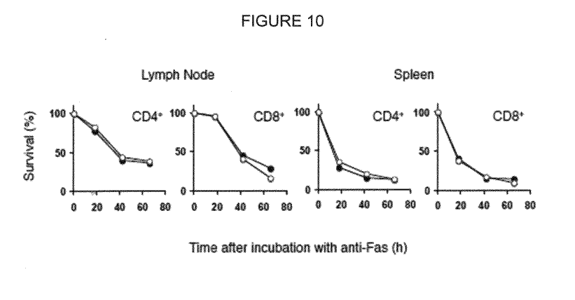

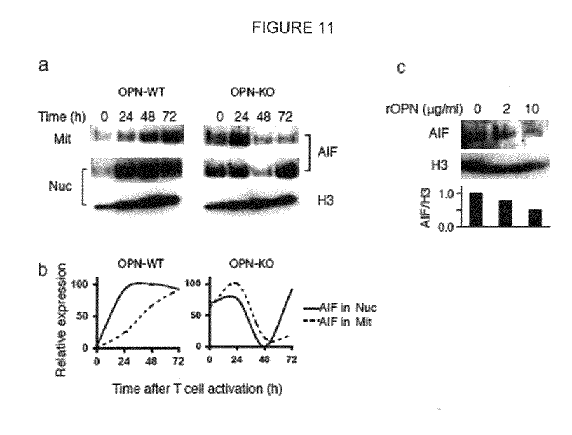

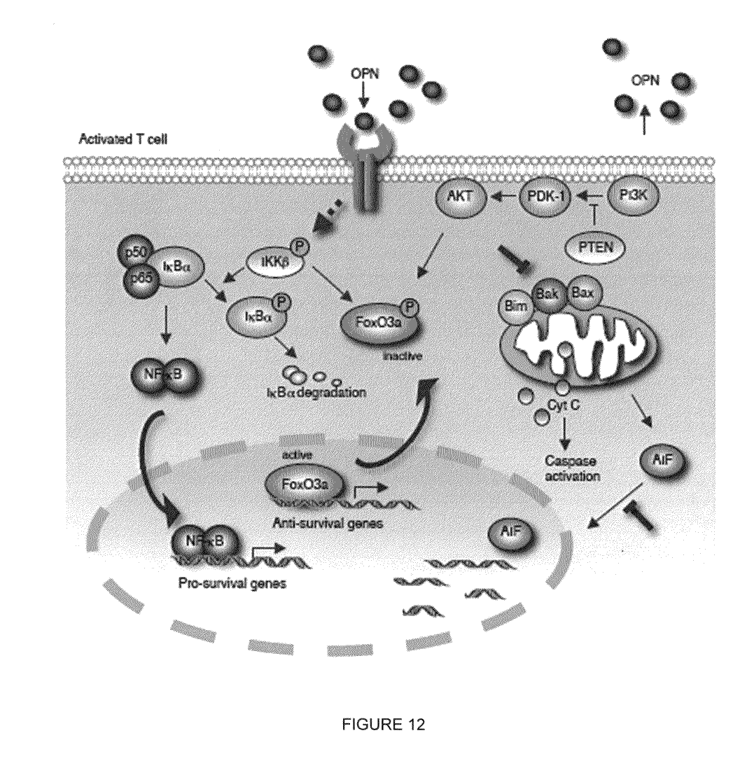

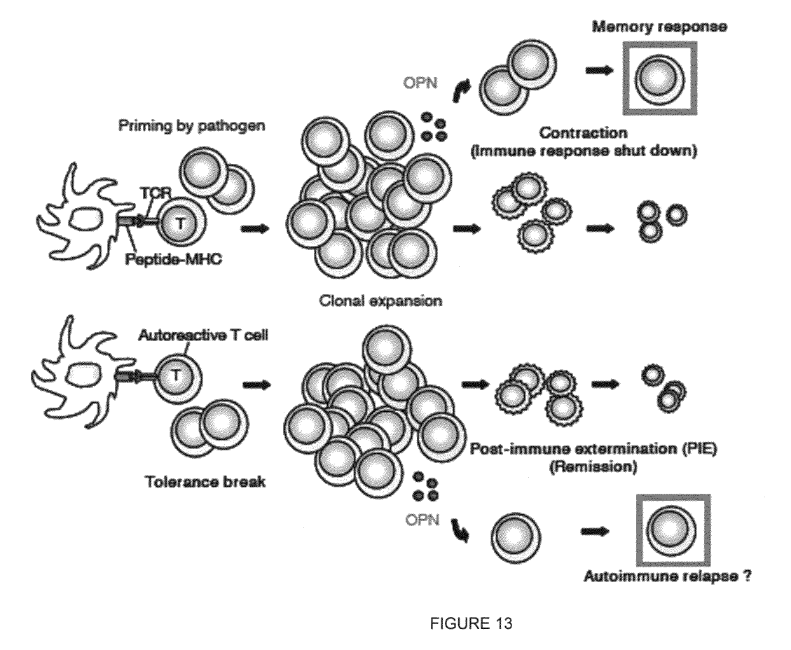

Furthermore, in this experiment all mice (6 out of 6) in the OPN-treated group developed eyelid swelling and tearing or atrophy of the eye, which are clinical signs of optic neuritis (Table 2, FIG. 6), whereas only 1 out of six mice in the control group developed these signs with a later onset. Optic neuritis is one of the hallmark presentations of MS and other related demyelinating diseases, including neuromyelitis optica. Given a previous report that the development of spontaneous autoimmune optic neuritis was observed in a proportion (35%) of older MOG-specific TCR transgenic mice at the approximate mean age of 6 months, the appearance of optic neuritis with higher incidence (100%) in these much younger mice that were five weeks old at the time of initial immunization, is likely due to the OPN administered over the course of primary and secondary immunizations with MOG peptide. Enhanced severity and accelerated onset of EAE with increased incidence of clinical optic neuritis accompanying OPN administration strongly suggest that OPN augments autoreactive T cell responses upon reactivation with MOG peptide. Taken together these experiments indicate that animals with elevated OPN concentrations are profoundly more susceptible to develop autoimmune CNS diseases when they are exposed to repeated activation of autoreactive T cells.

To address the potential contribution of endotoxin present in purified rOPN, we also administered endotoxin lipopolysaccharide (LPS), to the mice with EAE. The residual endotoxin concentration in recombinant rOPN we used was as analyzed as less than 0.003 EU per μg of purified protein in a standard Limulus amoebocyte lysate (LAL) assay. The clinical scores of SJL/J and MOG-specific TCR transgenic 2D2 mice with EAE receiving 0.5 ng LPS were not different from PBS-treated group. Thus, the residual LPS in purified protein does not contribute the effect of OPN on EAE described above.

Given that the apoptosis of infiltrating T cells in lesions has been observed during the clinical recovery in autoimmune diseases of the nervous system, including animal models of MS and Guillain-Barre syndrome we postulated that milder clinical symptoms in OPN-KO mice with EAE might be related to increased cell death of pathogenic lymphocytes infiltrating the CNS. We therefore examined cell death in affected tissues of EAE in OPN-WT and OPN-KO mice. Terminal deoxynucleotidyltransferase-mediated UTP end labeling (TUNEL) assays were performed on CNS tissue sections obtained during spontaneous clinical recovery exhibited in OPN-KO mice after the peak of disease. More TUNEL-positive cells were observed in the parenchymal inflammatory foci of the OPN-KO mice than OPN-WT mice with EAE (FIG. 1e) Thus absence of OPN results in enhanced cell death of leukocytes in situ.

These results demonstrate that OPN modulates apoptotic elimination of infiltrating lymphocytes occurring after an initial inflammatory autoimmune response in the CNS. The elimination of autoreactive T cells may be strongly inhibited by OPN in the CNS inflammatory lesions in vivo.

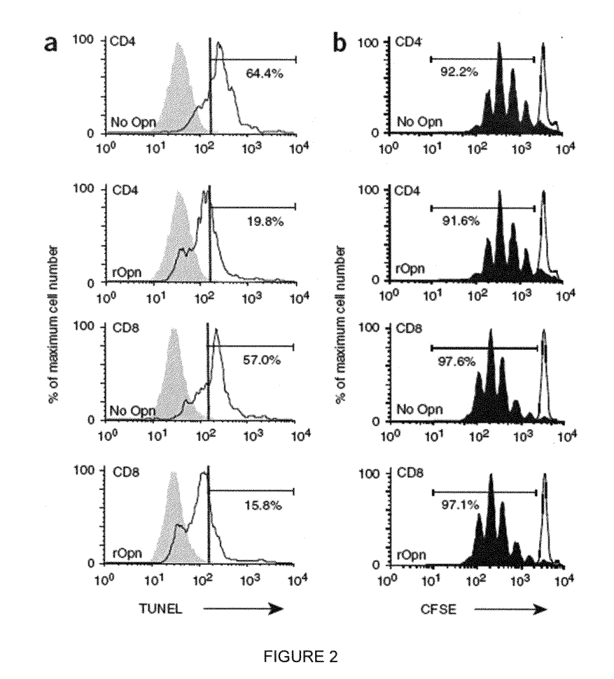

Enhanced survival of activated T cells by OPN. Next, the effect of OPN on activated T cell death was assessed in vitro by adding rOPN to the cultures of stimulated T cells. To monitor cell death we measured DNA fragmentation in the TUNEL assay. We found that OPN reduces the percentage of TUNEL-positive apoptotic T cells both in CD4⁺ and CD8⁺ T cell subsets after activation (FIG. 2a). We also observe that OPN added in culture increases the antigen-specific [³H] thymidine incorporation of CD4⁺ T cells specific for a pathogenic epitope of myelin basic protein, MBP Ac1-11. The increase in [³H] thymidine incorporation could be due to decreased cell death and/or increased cell division. To address these possibilities, we analyzed the cell division of activated T cells, using 5- (and 6-) carboxyfluorescein diacetate succinimidyl ester (CFSE)-labeled splenic T cells cultured with OPN. To minimize the effect of endogenously secreted OPN, cells from OPN-KO mice were used for this analysis. For both CD4⁺ and CD8⁺ T cells, the cell division profile and the percentage of cycling cells were not substantially altered by additional OPN (FIG. 2b), indicating that OPN does not enhance cell division. This finding was confirmed by a cell cycle analysis, using 5′-Bromo-2′-deoxyuridine (BrdU) and 7-Aminoactinomycin D (7-AAD) in which we obtained similar results. Thus, these results demonstrate that OPN inhibits T cell death without influencing cell division.

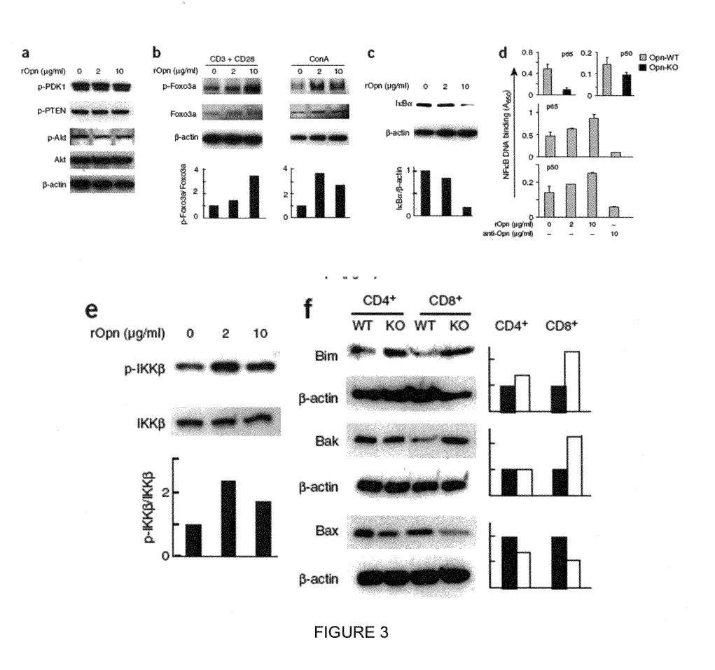

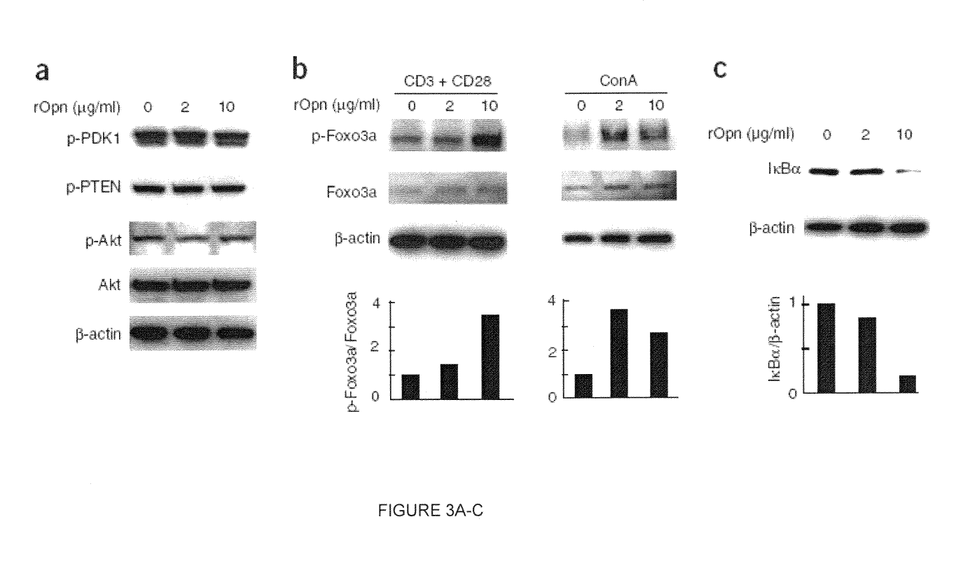

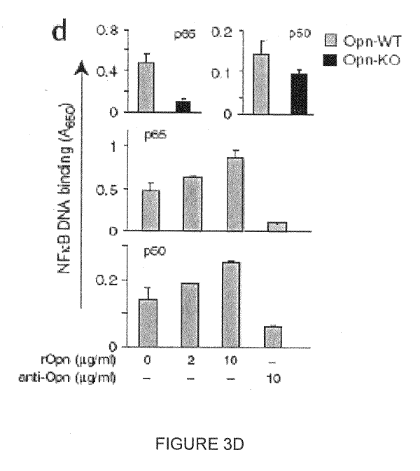

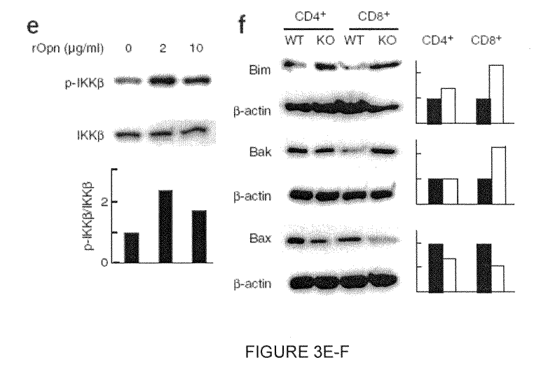

To further investigate the molecular mechanism underlying OPN-induced survival of activated T cells, we examined whether OPN affects the activity of proteins involved in cell survival in activated T cell signal transduction. It is noted that OPN induces phosphatidylinositol 3-kinase (PI3K) activity and Serine/Threonine kinase, Protein Kinase B/AKT (AKT) phosphorylation in breast cancer cell lines and in a pro-B cell line. We examined the activities of phosphoinsitide-dependent kinase 1 (PDK1), which activates AKT, and a negative regulator of PI3K and AKT signaling, tumour suppressor phosphatase and tensin homolog (PTEN) as well as AKT by assessing phosphorylation of these molecules in the presence of exogenously added OPN in the culture of activated T cells. Phosphorylation of PDK1, PTEN and AKT was not affected by added OPN in primary T cells activated by CD3 and CD28 stimulation (FIG. 3a). Although it is reported that OPN induces AKT phosphorylation in some tumor cell lines, AKT phosphorylation was not substantially increased in activated primary T cells by OPN, at least, examined in our experimental conditions.

Next, we further investigated whether OPN affects other downstream effectors involved in the P13K and AKT pathway. A Forkhead family transcription factor, Forkhead Box Class O 3a (FoxO3a), induces transcription of target genes that include proteins involved in DNA damage repair and apoptosis. Since FoxO3a is one of the downstream substrates of AKT and the transcriptional activity of FoxO3a is regulated by phosphorylation which serves as a nuclear exclusion signal, we analyzed the phosphorylation status of FoxO3a in the presence of OPN. An inactive, phosphorylated form of FoxO3a was increased in the presence of rOPN added in the culture. This strong phosphorylation of FoxO3a by OPN was observed in activated T cells both in CD3 plus CD28 stimulation and in Concanavalin A (ConA) stimulation (FIG. 3b). FoxO3a phosphorylation was more dose-sensitive to OPN in ConA stimulation. These results indicate that OPN inhibits the transcriptional activity of FoxO3a, which is a transcription factor inducing expression of pro-apoptotic genes.

We also investigated the impact of OPN on activation of transcription factor, nuclear factor kappa B (NF-κB) that plays important roles in multiple pathways including cell survival and induction of T_{H}1 cytokines. The activation of NF-κB represented by degradation of nuclear factor kappa B inhibitor alpha (IκBα) was strongly induced by OPN (FIG. 3c). The activation of NF-κB by OPN was also directly tested (FIG. 3d) for DNA binding of p65 and p50. OPN enhanced NF-κB activation and anti-OPN suppressed the activation of NF-κB in activated T cells.

We next examined whether OPN also affects the kinase upstream of IκBα, IkappaB kinase beta (IKKβ). Active IKKβ phosphorylates IκBα and induces degradation of phospho-IκBα. Immunoblot analysis revealed that IKKβ phosphorylation was increased in the presence of high concentrations of OPN within 24 h of T cell activation (FIG. 3e). Our results indicated that active NF-κB formation triggered by IκB degradation was induced by OPN via IKKβ phosphorylation. Furthermore, phosphorylated, active IKKβ is known to phosphorylate FoxO3a as well. Thus it is likely that both increased phosphorylation of FoxO3a and degradation of IκBα were mediated by IKKβ in OPN signaling.

Taken together, these results indicate that OPN inhibits the transcriptional activity of FoxO3a by increasing phosphorylation, while at the same time it induces activation of NF-κB in activated T cells. It is intriguing that both downstream events of OPN signaling can be mediated by active IKKβ and contribute to the pro-survival role of OPN in T cells since the target genes of FoxO3a include pro-apoptotic proteins such as Bim and the target genes of NF-κB include numerous pro-survival proteins.

It has been reported that a group of proteins designated “BH3-only Bcl-2 family proteins”, including Bim (Bcl-2 interacting molecule), and another group of proteins designated “multi-BH domain proteins”, including Bak and Bax, play critical roles in cell death, driving mitochondrial dysfunction. Bim activates Bak and Bax, and this activation is hindered by Bcl-2 or Bcl-x_{L}, the pro-survival Bcl-2 family members, which directly bind to Bim. Therefore, in this pathway, cell death is likely to be regulated by the quantitative balance between pro- and anti-apoptotic Bcl-2 family proteins.

Bim is known as one of the target genes of FoxO3a, which we observed is regulated by OPN in the present experiments. We next, examined the expression of Bcl-2 family proteins in the OPN-KO T cells. Interestingly, Bim is expressed at a higher level in resting OPN-KO CD4⁺ and CD8⁺ T cells than in OPN-WT counterparts. Moreover, the expression of both Bak and Bax was also altered in the OPN-KO T cells (FIG. 3f).

We further examined the expression profiles of these proteins induced upon T cell activation. The expression of Bim and Bak was upregulated upon T cell activation over a 72-hour period, whereas Bax expression was only slightly changed over time. The expression kinetics derived from the immunoblot analysis revealed that the expression profiles of these proteins upon T cell activation were strikingly altered in the OPN-KO compared to the OPN-WT. In the OPN-KO mice, the elevated Bim expression at the resting stage diminished over the 48-hour period of T cell activation but Bim expression was induced again after that time. A similar trend was observed in Bak expression, except that re-induction during a later time period was not observed. The expression of Bax exhibited a substantial change in the OPN-KO as well, with a lower expression at the resting stage and a greatly induced expression after 48 hours. Collectively, this result suggests that OPN modulates the expression of the pro-apoptotic proteins, Bim, Bak and Bax. We also examined the expression of the anti-apoptotic proteins, Bcl-2 and Bcl-x_{L}; however, we were unable to detect distinct changes in the OPN-KO compared to the OPN-WT.

We next examined Fas-mediated cell death of the OPN-KO T cells using Fas antibodies in vitro. Flow cytometric analysis revealed that the apoptosis of OPN-KO T cells induced by Fas crosslinking is comparable to the OPN-WT T cells in both CD4⁺ and CD8⁺ subpopulations. In concordance, the OPN-KO T cells exhibited little or no change in Fas and FasL expression on their surfaces compared to the OPN-WT. This result implies that OPN does not affect the Fas-mediated death of activated T cells.

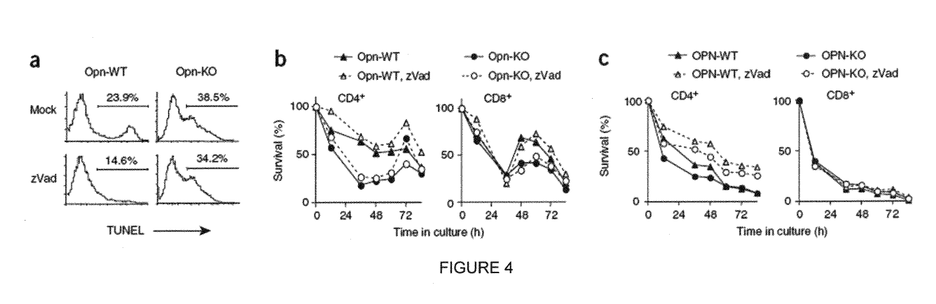

Mode of cell death inhibited by OPN. Massive caspase activation is frequently found in apoptosis, often associated with mitochondrial instability. We tested whether enhanced survival of T cells by OPN is associated with inhibition of Caspase activation. We explored this issue using a caspase inhibitor, z-Vad in the TUNEL assay to examine activated T cell death. Activated T cells cultured with z-Vad revealed that the inhibition of caspases was not sufficient to rescue activated CD4⁺ T cells from the heightened cell death in the OPN-KO (FIG. 4a). In the kinetic study of T cell survival after activation, OPN deficiency, which was associated with profound cell death in CD4⁺ T cells, began at an early time period, whereas OPN-WT CD4⁺ T cells appear more resistant to this early death. In addition, caspase dependency was more profound for cell death in CD4⁺ than in CD8⁺ T cells of OPN-WT mice (FIG. 4b). Collectively, these results suggested that OPN-KO T cells were predisposed to death, more prominently in CD4⁺ T cells, and that the cell death occurred in a caspase-independent manner, modulated via OPN.

We also found that OPN deficiency differentially influences the viability of resting CD4⁺ and CD8⁺ T cells (FIG. 4c). Resting OPN-KO CD4⁺ T cells were less viable than WT CD4⁺ T cells without stimulation in culture, whereas resting CD8⁺ T cell death was not affected by OPN deficiency. Interestingly, survival of resting. CD4⁺ T cells was increased by caspase inhibition in both OPN-KO and OPN-WT cells indicating that the activity of caspase is important in this mode of spontaneous cell death of resting T cells. These observations suggest that OPN also promotes the survival of naïve CD4⁺ T cell but does not affect naïve CD8⁺ T cells.

Release of mitochondrial proteins is observed as a result of mitochondrial dysfunction in different modes of cell death. Apoptosis inducing factor (AIF), a flavoprotein located in mitochondria is released from mitochondria under variety stimulus inducing cell death. AIF has been reported to have a dual function in cell survival. It should be noted that while there are several recent publications indicating that AIF plays an important role in apoptosis and even in apoptosis of oligodendrocytes, some investigators indicate that AIF may not play a critical and direct role in apoptosis, but may merely be a surrogate marker for biochemical changes associated with this process.

Those who support an important physiological role of AIF in apoptosis, demonstrate that when localized in mitochondria, the oxidoreductase activity of AIF protects cells from oxidative stresses, promoting cell survival; however, nuclear translocation of AIF occurs upon cell death, and is associated with chromatin condensation and massive DNA fragmentation.

The effect of OPN on the nuclear translocation of AIF in activated T cell death was examined using the OPN-deficient splenocytes and rOPN added in culture. Subcellular fractionation and immunoblot analysis revealed that the nuclear localization of AIF was greatly increased in the early phase of T cell activation in OPN-KO cells. We found that AIF expression in the nucleus was induced after T cell activation in the OPN-WT splenocytes. In contrast, in the OPN-KO mice the nuclear AIF abundance was constitutively elevated in the resting state and maintained until 24 hours after activation. This elevation was observed in the mitochondria as well, demonstrating that the regulation of AIF expression was impaired in OPN-KO splenocytes. We also found that the temporal decline of nuclear AIF is rapidly recovered in OPN-KO cells within 24 hours. This observation suggests that OPN deficiency promotes the expression and nuclear localization of AIF. The marked inhibitory effect of OPN on nuclear translocation of AIF was also directly confirmed using recombinant OPN (rOPN) in culture of activated T cells. Whether more nuclear localization of AIF sensitizes OPN-KO T cells to programmed cell death, or whether the action of OPN in regulating the nuclear versus mitochondrial location of AIF merely reflects AIF's trafficking and sub-cellular localization as a surrogate marker of apoptosis, it is clear that OPN influences the localization of this protein.

Thus, the provided cellular and biochemical studies collectively demonstrate that OPN plays a critical role in T cell survival by rescuing those cells from programmed cell death induced by T cell activation. First, OPN prevents T cell death by regulating the activity of transcription factors FoxO3a that induces genes including pro-apoptotic proteins. Second, OPN promotes cell survival by inducing the NF-κB activation. Third, OPN affects the expression of pro-apoptotic Bcl-2 proteins, Bim, Bak and Bax.

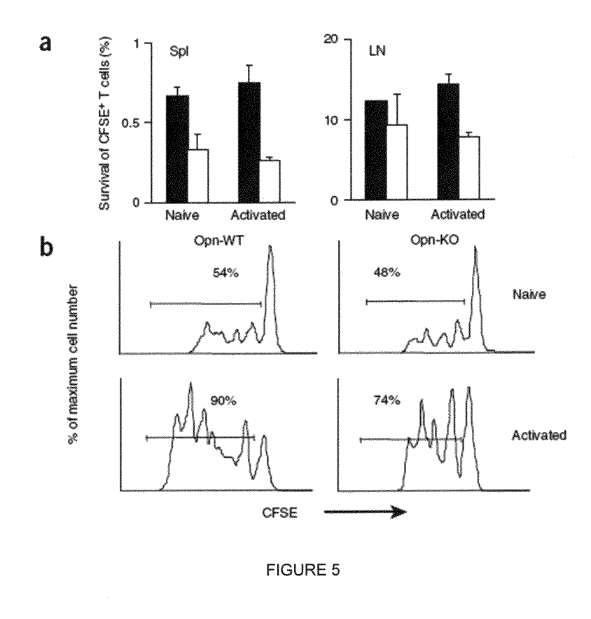

Post-immune extermination of activated T cells and OPN. Finally, the pro-survival activity of OPN was confirmed in vivo via adoptive transfer experiments. CFSE-labeled T cells from the OPN-KO and OPN-WT mice were transferred to lymphopenic recipients (Rag1^{-/-} mice), and the survival of transferred T cells was analyzed using flow cytometry on day 8 post transfer. Consistent with our other results, the OPN-KO T cells had a lower survival rate than the OPN-WT T cells in the Rag1^{-/-} host (FIG. 5a). Notably, this reduced survival rate was more prominent when activated T cells, rather than naïve T cells, were transferred suggesting that survival of T cells was sensitive to OPN to a greater degree in activated T cells in vivo (FIG. 5a,b). The importance of the OPN produced from different sources was also addressed by this transfer experiment. The decreased survival of OPN-KO T cells in the Rag1^{-/-} host, where the other sources of OPN, such as macrophages, can produce OPN normally, indicates that OPN secreted from T cells is critical in T cell survival and may function in an autocrine or paracrine fashion.

Here we show that the mice in three different models of EAE, when treated with OPN, exhibit more severe clinical courses associated with worsening of disease, and more severe relapses culminating in death. An overall profile of disease progression of these mice is consistent with secondary progressive MS (SPMS) that is characterized by steady disease progression. Most relapsing-remitting MS (RRMS) patients develop the secondary progressive phase through recurring relapses. In SPMS, although isolated attacks are often accompanied by spontaneous remissions, the clinical recoveries are incomplete, and gradual exacerbation of the symptoms appears between attacks. The underlying mechanism for the progression of the disease through recurrent relapses is one of the key unanswered questions in research on autoimmune diseases. Our results here demonstrating the surprising potency of OPN in autoimmune disease along with OPN-induced T cell survival teach that inflammatory autoimmune responses can be quenched by programmed cell death of autoreactive cells during spontaneous remission. This process is inhibited by OPN secreted from activated T cells, especially in MS patients who have highly elevated OPN amounts in brain lesions and in plasma prior to and during relapse. Given that OPN mediates the clinical relapse in an EAE model, here we propose a potential model for autoimmune relapse. The apoptotic extermination of autoreactive T cells activated as a result of a break in tolerance in certain conditions may occur as an alternative or an extra layer of the protection from severe autoimmunity. The effective “post-immune extermination (PIE)” of autoimmune T cells may contribute to minimizing the risks of secondary autoimmune responses. In our model, the pro-survival role of OPN may provoke the incomplete PIE of autoreactive T cells, leading to relapses in autoimmune diseases. The process of post-immune extermination of self-reactive T cells could provide an important control measure to remove pathogenic T cells in order to limit the tissue damage. Therefore, it appears that T cell death may play a key role, not only in preventing autoimmunity through self-tolerance, but also in extinguishing autoimmune activation after disease is initiated. One of the remarkable features of autoimmune diseases is their chronicity, often lasting the entire adult life of the patient.

Our findings suggest that high OPN expression actively exacerbates relapse and progression of autoimmune disease. We also have identified OPN as a critical cytokine that saves activated T cells from death. We found that a Forkhead family transcription factor, FoxO3a, and NF-κB were reciprocally regulated by OPN. Following an OPN signal, highly phosphorylated FoxO3a was excluded from the nucleus and inactivated. In contrast, OPN increased IκBα degradation, triggering transcriptional activation and nuclear translocation of NF-κB. The finding that OPN activated IKKβ provides a strong mechanistic basis to explain control of both transcription factors in response to OPN. This dynamic regulation of FoxO3a and NF-κB controls the balance between death and survival of activated T cells.

In our results, OPN alters the expression of pro-apoptotic proteins Bim, Bak and Bax. The expression kinetics of these proteins in OPN-KO are also different from OPN-WT. Bim, Bak and Bax expression kinetics were differentially regulated in OPN-KO under our experimental conditions. Consistent with our results, the sequential and differential modulation of Bak and Bax has been reported by others in various different conditions of apoptosis such as drug-induced apoptosis of cancer cells, IL-12 treated T cells and TRAIL-induced apoptosis of leukemic cells, as well. The data show that a cytosolic protein Bax is activated and translocated to mitochondria later than the activation of Bim and Bak which are located in mitochondria in T cells supported the concept that Bak may be more critical than Bax in activated T cell death, at least in the initiation of the process. The differential expression and involvement of Bak and Bax in various environments where cells are dying, along with our present observations, imply that they have differing physiological roles in apoptosis. Bax expression is induced at a later time point (48 h) than Bak, which is upregulated in resting and early stage of T cell activation in OPN-KO. This observation can explain the bi-phasic characteristic of the cell survival curve of activated T cells in our results. That is, higher expression of Bim and Bak in resting OPN-KO cells contributes to enhanced and accelerated cell death of OPN-KO in an early stage and the subsequent induction of Bax in OPN-KO contributes to cell death at a later stage. The higher abundance of Bax in 48 h in OPN-KO than OPN-WT may compensate for the lower abundance of induction of Bim and Bak in the same time period. These kinetic studies imply that OPN-KO T cells are predisposed to death in association with increased abundance of Bim and Bak in the resting stage. These findings further imply that this enhanced cell death at a later stage would result in less survival of activated autoreactive T cells in vivo with resolution of relapses of EAE.

The OPN-induced signaling pathway, resulting in increased survival of activated T cells, appears to influence the caspase-independent death pathway whereas the Fas-mediated death pathway is not affected by OPN. Consistent with our results, it was reported that costimulation through αᵥβ₃ integrin by OPN in vitro mediates IL-2 secretion without inducing programmed cell death of T cells and furthermore, the OPN effect was shown to be independent of caspase pathways and of Fas-mediated pathways in recent studies using cardiac fibroblasts and human PBMCs, respectively. Collectively, our findings emphasize the potential importance of the cytokine-mediated regulation of the cell autonomous death pathway in T cells.

There is indirect evidence for a role of OPN in spontaneous recovery from autoimmune diseases in other models. Of interest are studies on knock out mice for the CD44 isoform v7. CD44 v7 is one of the receptors for OPN. CD44 v7-deficient mice and both CD44 v6 and v7-deficient double transgenic mice showed a complete spontaneous recovery in experimental colitis similar to the spontaneous recovery observed in OPN-KO mice in EAE. Experimental colitis is an animal model of human inflammatory bowel diseases, such as Crohn's disease, in which OPN is also upregulated¹⁰. More importantly, increased cell death of leukocytes was observed in inflamed regions of bowel in these mice, with CD44 gene deletions. Hence, OPN receptor-deficiencies show increased cell death of leukocytes that infiltrate the target tissue and spontaneous recovery in tissue specific autoimmune diseases, like EAE and experimental colitis.

Our findings not only demonstrate the anti-inhibitory role of OPN in activated T cell death and its underlying mechanisms, but also reveal the role of OPN in the relapse and progression of an autoimmune disease. This study may provide new insights into the regulation of T cell viability as they support the idea of an additional layer of protection in the immune system to mitigate autoimmune attacks. Furthermore, our results provide a therapeutic target for the treatment of perplexing autoimmune relapses, which can be applicable to many autoimmune diseases.

TABLE 2
Severe EAE and autoimmune optic neuritis induced by OPN in MOG-specific
TCR transgenic mice

| | EAE | | | | Optic neuritis | |
|---|---|---|---|---|---|---|
| | | | Mean day of | Mean maximal | | Mean day of |
| Treatment^{a} | Incidence | Mortality | onset^{b} | score^{c} | Incidence | onset^{b} |
| rOPN | ^{d}5/6 (83%) | ^{e}4/6 (67%) | 9.0 ± 0.9** | 4.0 ± 0.8 | ^{f}6/6 (100%) | 15.7 ± 2.1 |
| PBS | 4/6 (67%) | 0/6 (0%) | 14.3 ± 0.3** | 1.8 ± 0.6 | 1/6 (17%) | 17.0 ± 0.0 |

^{a}MOG-specific TCR transgenic mice were immunized with MOG peptide and injected with either rOPN or PBS as described in FIG. 1c.
^{b}Onset of disease was determined by days from the secondary MOG peptide immunization. Data are presented as mean ± S.E.M. ANOVA test, **p = 0.0017
^{c}Data are presented as mean maximal clinical scores for the animals showing clinical disease.
^{d}Number of mice developed EAE/n
^{e}Number of mice died from EAE/n
^{f}Number of mice developed optic neuritis/n

### Methods

Mice. Female SJL/J, C57BL/6 and Rag1-deficient mice (8-12-weeks old) were purchased from the Jackson Laboratory. MBP Ac 1-11 TCR transgenic mice were previously described⁵⁰. OPN-KO and OPN-WT mice in 129/C57BL/6 mixed background were previously described⁴. OPN-KO C57BL/6 background (backcrossed 11 generation) mice were used and for OPN-WT C57BL/6 background, wild type C57BL/6 mice were used as control purchased from the Jackson Laboratory. MOG-specific TCR transgenic mice (2D2) were backcrossed into the C57BL/6 background²³. All animal protocols were approved by the Division of Comparative Medicine at Stanford University and the Committee of Animal Research at the University of California San Francisco, in accordance with the National Institutes of Health guidelines.

Peptides. MOG p 35-55 (MEVGWYRSPFSRWHLYRNGK), PLP p 139-151 (HCLGKWLGHPDKF) were synthesized on a peptide synthesizer (model 9050; MilliGen) by standard 9-fluorenylmethoxycarbonyl chemistry, and purified by high-performance liquid chromatography (HPLC). Amino acid sequences were confirmed by amino acid analysis and mass spectroscopy. The purity of each peptide was greater than 95%.

Induction of experimental autoimmune encephalomyelitis. EAE was induced in SJL/J mice with PLPp 139-151. In C57BL/6 mice, OPN-KO and OPN-WT mice and MOG-specific TCR transgenic mice, EAE was induced by immunization with 100 μg of MOG p 35-55. All peptides were dissolved in complete Freund's adjuvant (CFA) containing 4 mg/ml of heat-killed Mycobacterium tuberculosis H37Ra (Difco Laboratories) as described in^{4,50}. On the day of immunization and 48 h later, C57BL/6 mice, OPN-KO and OPN-WT mice and MOG p 35-55 TCR transgenic mice were injected with 50 ng of Bordetella pertussis toxin (PT) in PBS, intravenously (i.v.). Mice were examined daily for clinical signs of EAE and scored as follows: 0, no paralysis; 1, loss of tail tone; 2, hind limb weakness; 3, hindlimb paralysis; 4, hindlimb and forelimb paralysis; 5, moribund or dead. For an individual mouse, a remission was defined by a decrease of the score of at least one point for at least two consecutive days. Assessment of Optic Neuritis: MOG-specific TCR transgenic mice can develop spontaneous optic neuritis. These mice were examined daily for assessment of clinical signs including eyelid redness and swelling and tearing and atrophy of the eye.

Administration of recombinant OPN in vivo. OPN-KO mice with EAE were treated with 5 μg carrier-free recombinant mouse OPN (R&D) suspended in 100 μl PBS by injection into the tail vein. Residual endotoxin concentration in recombinant mouse OPN was assessed by the Limulus amoebocyte lysate (LAL) test (Sigma). The amount of endotoxin was less than 0.003 EU per microgram OPN. PBS was administered i.v. as a control. Treatment was begun during the first remission of each individual mouse and given daily.

TUNEL assay of tissue sections and histopathology. Mice with EAE were sacrificed 8 to 10 days after the onset of the disease. Brains and spinal cords were fixed in 4% (w/v) paraformaldehyde and embedded in paraffin. TUNEL-positive cells were detected in deparaffinized sections using an Apoptag Plus Detection Kit (Intergen Co.) according to the manufacturer's instructions. The 3,3′-diaminobenzidine (DAB)-stained sections were fixed with 10% neutral buffered formalin in PBS and counterstained with hematoxylin for light microscopy.

TUNEL assay of peripheral lymphocytes. Terminal deoxynucleotidyltransferase-mediated UTP End Labeling (TUNEL) assay was performed using an apoptosis detection system, the In Situ Cell Death Detection Kit (Roche Molecular Biochemicals) according to the manufacturer's instructions. Briefly, lymph node cells and splenocytes were stimulated and cultured for various time periods. In some cases, 100 mM z-VAD-fmk (BD PharMingen), a pan-Caspase inhibitor, was added to the culture. Cells were fixed in 2% paraformaldehyde for 1 h at 25° C. and then permeabilized in 0.1% Triton-X 100 in 0.1% sodium citrate for 2 min at 0° C. Samples were then incubated at 37° C. in the dark for 1 h in the TUNEL reaction mixture containing terminal deoxynucleotidyl transferase (TdT) and FITC-dUTP. Labeled DNA strand breaks in apoptotic cells (TUNEL⁺ cells) were analyzed using FACS.

CFSE labeling. Single cell suspensions made from spleens and lymph nodes or from column-purified T cells were suspended at 4×10⁷ cells/ml in phosphate-buffered saline (PBS) containing 5% fetal calf serum (FCS). 5- (and 6-) carboxyfluorescein diacetate succinimidyl ester (CFSE) (Molecular Probes) diluted in PBS containing 5% FCS was added to an equal volume of pre-warmed cell suspension at a final concentration of 5 μM; the suspension was mixed rapidly. The cells were incubated at 37° C. for 15 min and then centrifuged (300×g) at room temperature for 5 min. The pellet was resuspended in the culture medium (see above; T cell activation and proliferation assay) and incubated at 37° C. for an additional 30 min. At the end of the incubation, the cells were washed three times in PBS containing 5% FCS and resuspended in the culture medium.

T cell activation. Splenocytes and lymph node cells were isolated from OPN-WT, OPN-KO or MBP TCR transgenic mice and were resuspended in a culture medium consisting of RPMI 1640 supplemented with L-glutamine (2 mM), sodium pyruvate (1 mM), non-essential amino acids (0.1 mM), penicillin (100 U/ml), streptomycin (0.1 mg/ml), 2-mercaptoethanol (50 μM) and either 10% FCS for the MBP TCR transgenic cells or 1% autologous normal mouse serum for the OPN-WT and the OPN-KO cells. The T cells isolated from the spleen and lymph nodes were stimulated with ConA (2 μg/ml) and cultured for various time periods. For T cell stimulation using anti-CD3 and anti-CD28, lymph node cells (2×10⁶ cells/ml), or CD3⁺ T cells (2×10⁶ cells/ml) purified by negative selection (columns from R&D Systems) were stimulated in 6-well plates previously coated with 5 μg/ml of both anti-CD3 (clone 145-2C11; BD Biosciences) and CD28 (clone 37.51; BD Biosciences) or 5 μg/ml of anti-CD3 alone.

Flow cytometry. Immunofluorescent staining of cells for FACS was performed using standard protocol. Briefly, 5 to 10×10⁵ cells were suspended in a FACS buffer (PBS with 2% FCS) and stained at 25° C. for 20 min. Fluorochrome-conjugated monoclonal antibodies (anti-CD4-fluorescein isothiocyanate (FITC), -phyto-endorphin (PE), -peridinin-chlorophyll-protein (PerCP); anti-CD8-FITC, -PE, -PerCP; anti-CD3ε-FITC, -PE; anti-TCR V_{β}8-PE) were all purchased from BD PharMingen. For biotinylated antibodies (anti-TCR V_{β}6, anti-TCR V_{β}12), the FITC- or PE-conjugated Streptavidin (BD PharMingen) was used. Stained cells were washed three times and analyzed using a FACScan cytometer, CellQuest software (Becton Dickinson) and Flowjo software (Tree Star). To assess cell viability, 1 μg/ml propidium iodide (PI) was added. 5×10⁴ to 10⁵ events were analyzed.

Subcellular fractionation and immunoblot analysis. Nuclear and mitochondrial fractions were obtained by differential centrifugation. Mouse lymph node cells and spienocytes (5×10⁶ cells) were washed with ice-cold PBS and resuspended in 200 μl of ice-cold isotonic homogenization buffer (250 mM sucrose, 10 mM KCl, 1.5 mM MgCl₂, 1 mM Na-EDTA, 1 mM Na-EGTA, 1 mM dithiothreitol, 0.1 mM phenylmethylsulfonylfluoride, 10 mM Tris-HCl, pH 7.4) containing a proteinase inhibitor cocktail (Roche). Cells were broken by 80 strokes in a pre-chilled Dounce homogenizer; then the homogenates were spun down at 30×g for 5 min to remove unbroken cells. The nuclei were pelleted by the centrifugation of the supernatant at 750×g for 10 min. For the mitochondrial fraction, the supernatants were re-centrifuged at 14,000×g for 20 min. Pellets from the nuclei were washed three times with a homogenization buffer containing 0.01% NP-40. The lysate from 10⁶ cells per lane was used for SDS-PAGE, and immunoblot analysis was performed using the ECL detection system (Amersham BioSciences, Inc.). Antibodies for immunoblots were purchased from the following sources: Anti-phospho-PDK1, anti-phospho-PTEN, anti-phospho-FoxO3a and anti-FoxO3a antibodies (Cell signaling); Anti-Bim (BD PharMingen); anti-Bak and anti-Bax (Upstate); anti-Caspase-3 and anti-histone H3 (Cell signaling); anti-β-actin (sigma); anti-AIF (Santa Cruz). The quantification of the proteins detected on immunoblots was performed by measuring band intensity with ImageJ software (National Institutes of Health). The band intensity of a protein was normalized to that of β-actin taken in the same blot. NF-κB activation was measured by DNA binding of p65 and p50 using NF-κB ELISA kit. (Clontech).

Statistical analysis. Data are presented as mean±s.e.m. The statistical significance was analyzed using a one-way multiple-range analysis of variance test (ANOVA) for multiple comparisons or Fisher exact probability test (one-tailed). A value of P<0.05 was considered to be significant.

TABLE 3
EAE induced in OPN-KO and OPN-WT mice

| | | | | Mean maximal | |
|---|---|---|---|---|---|
| | | | Mean day of | score after | Mean day of |
| Treatment | Incidence | Mortality | onset^{b} | treatment | injection^{c} |
| WT | 100% (10/10)^{d} | 20% (2/10)^{e} | 8.5 ± 0.5 | 4.0 ± 0.2 | N/A |
| OPN-KO, PBS | 100% (7/7) | 14% (1/7) | 11.0 ± 0.8 | 2.5 ± 0.3 | 17 ± 0.4 |
| OPN-KO, | 100% (7/7) | 100% (7/7) | 10.0 ± 0.9 | 5.0 ± 0.0 | 17 ± 0.8 |
| rOPN^{a} | | | | | |

^{a}Recombinant (r)OPN 5 μg/mouse daily
^{b}Data are mean ± s.e.m.
^{c}Mean first day of rOPN or PBS injection
^{d}(number of mice developed EAE/n)
^{e}(number of mice died from EAE/n)

### EXAMPLE 2

### Treatment of Relapsing Multiple Sclerosis and Other Autoimmune Diseases with Anti-Osteopontin Antibodies

Strains of mice susceptible to chronic relapsing EAE (for example, SJL mice) are induced to develop EAE (for example, with PLPp 139-151 in complete Freund's adjuvant) on Day 1 of the experiment. The mice developed EAE at Day 10, and were scored with the standard scoring system: 1 for a limp tail, 2 for weak hind leg, 3 for paralyzed hind leg, 4 for weak fore leg. At day 14, the sick mice were split into 3 equal groups by score (randomization). Each group was given their respective treatment intravenously: vehicle or one of 2 osteopontin antibodies. The mice were given 0.2 mg of antibody every 3 days, and scored daily. Relapses generally occur every 2-3 weeks, and the animals are monitored over that time period.

Further analysis looks for differences in the number of brain and spinal cord lesions by pathology in the three groups, inflammation in the brain and spinal cord by checking the type and number of infiltrating T cells and other immune cells and their reactivity; and also to test for memory activity by rechallenging the spleen cells with PLP.

In humans that have been previously diagnosed with multiple sclerosis, osteopontin therapy is initiated following diagnosis. Antibodies specific for osteopontin are administered weekly or semi-weekly. Efficacy is demonstrated based on the reduction in the number and size of brain lesions (as measured by MRI scanning), the reduction of the number of disease relapses (episodes of clinical paralysis), and the slowing of progression to disability.

What is claimed is:

1. A method for inhibiting or preventing relapse of an autoimmune disease in a mammal, the method comprising:
administering to said mammal a therapeutically effective dose of an agent that inhibits osteopontin, wherein immune cells in tissues affected by the autoimmune disease have decreased survival in the presence of the agent.

2. The method of claim 1, wherein the mammal is diagnosed as having the autoimmune disease prior to the administering step.

3. The method according to claim 1, wherein said autoimmune disease is a relapsing demyelinating disease.

4. The method according to claim 2, wherein said disease is multiple sclerosis.

5. The method according to claim 1, wherein said agent is an antibody specific for osteopontin.

6. The method according to claim 1, wherein said agent is a nucleic acid that inhibits osteopontin expression.

7. The method of claim 1, further comprising:
monitoring the survival of activated T cells in tissues affected by the autoimmune disease.

8. The method of claim 1, further comprising:
monitoring the expression of transcription factors, FoxO3a and NF-κB in activated T cells in tissues affected by the autoimmune disease.

9. The method of claim 1, further comprising:
monitoring the level of osteopontin in a cell of the mammal selected from the group consisting of a neuron, a macrophage, a vascular endothelial cell, an astrocyte and a microglial cell.

10. The method of claim 1, wherein the mammal is a mouse or a human.
